(19) United States
(12) Patent Application Publication
(10) Pub. No.: US 2025/0213447 A1
(43) Pub. Date: Jul. 3, 2025

(54) COSMETIC COMPOSITION

(71) Applicant: SHISEIDO COMPANY, LTD., Chuo-ku, Tokyo (JP)

(72) Inventors: Ryushi FUKUHARA, Tokyo (JP); Kuanting LIN, Tokyo (JP); Kei UJIMOTO, Tokyo (JP)

(73) Assignee: SHISEIDO COMPANY, LTD., Chuo-ku, Tokyo (JP)

(21) Appl. No.: 18/879,427

(22) PCT Filed: Jul. 14, 2023
(86) PCT No.: PCT/JP2023/026019

§ 371 Date: Dec. 27, 2024

(30) Foreign Application Priority Data
Jul. 28, 2022 (JP) 2022-120587

(51) Int. Cl. A61K 8/37; A61K 8/06; A61K 8/27; A61K 8/29; A61K 8/891; A61Q 17/04

(52) U.S. Cl. CPC A61K 8/375; A61K 8/064; A61K 8/27; A61K 8/29; A61K 8/891; A61Q 17/04; A61K 2800/623; A61K 2800/651

(57) ABSTRACT

To provide a cosmetic composition having high water resistance even when no silicone oil is used or only a small amount thereof is used.

A cosmetic composition includes the following component: UV scattering agent particles, bis-diglyceryl polyacyladipate-2, and a liquid oil, and a content of a silicone oil in the liquid oil is 10 mass or less.

### TECHNICAL FIELD

The present invention relates to a cosmetic composition.

### BACKGROUND ART

In cosmetics having a UV protection effect, such as sunscreens, generally, metal oxide particles such as titanium oxide particles and zinc oxide particles are used as UV scattering agent particles. Such UV scattering agent particles are dispersed in a liquid oil and then added to cosmetics in many cases.

In addition, in order to improve a feeling of use, water resistance and the like of such cosmetics, a silicone oil is generally incorporated as a liquid oil.

For example, PTL 1 discloses a sunscreen cosmetic which has water resistance, imparts a satisfactory and sustainable UV protection effect, and can be easily washed off with soap or the like. More specifically, the sunscreen cosmetic of PTL 1 is an emulsion type sunscreen cosmetic containing a UV absorbing agent and/or a UV scattering agent, an organically modified clay mineral, a nonionic surfactant, a specific acrylic-silicone graft copolymer, an oil, and water, and in particular, volatile silicone is preferably used as the oil.

PTL 2 discloses a water-in-oil type emulsion cosmetic that contains the following (A) to (D): (A) bis-diglyceryl polyacyladipate-2, (B) an oil containing (b1), (b1) a volatile oil that has poor compatibility with (A), (C) an emulsifying agent, and (D) an aqueous component.

In addition, PTL 3 discloses a liquid composition for skin makeup (but not including boron nitride), which contains, in a physiologically acceptable medium, (a) at least one paste-like substance having a molecular weight of 1,000 or more and a melting point of lower than 50° C., (b) at least one silicone elastomer, (c) at least one pigment, (d) at least one filler, and (e) at least one dimethicone, wherein the (a) paste-like substance is selected from among bis-diglyceryl polyacyladipate-2, dimer dilinolic acid dimer dilinoleyl bis (behenyl/isostearyl/phytosteryl), and a mixture thereof, and the amount of the (a) paste-like substance is more than 1.0 mass % based on a total mass of the composition.

### CITATION LIST

### Patent Literature

[PTL 1] Japanese Patent Application Publication No. 2003-277248

[PTL 2] Japanese Patent Application Publication No. 2012-206986

[PTL 3] Japanese Patent No. 7013112

### SUMMARY OF INVENTION

### Technical Problem

In recent years, there has been demand for cosmetic compositions that are silicone-free or in which a reduced amount of silicone is used.

However, there is a problem in that, when no silicone oil or only a small amount thereof is used, it is difficult to obtain a cosmetic composition having sufficient water resistance.

The present invention has been developed in order to improve the above situation, and an object of the present invention is to provide a cosmetic composition having high water resistance even when no silicone oil or only a small amount thereof is used.

### Solution to Problem

The present invention which achieves the above object is as follows.

### Aspect 1

A cosmetic composition including the following components:
UV scattering agent particles,
bis-diglyceryl polyacyladipate-2, and
a liquid oil,
wherein a content of a silicone oil in the liquid oil is 10 mass % or less.

### Aspect 2

The composition according to Aspect 1,
wherein the UV scattering agent particles are a combination of surface-treated zinc oxide particles and surface-treated titanium oxide particles.

### Aspect 3

The composition according to Aspect 1,
wherein the surface-treated zinc oxide particles are one, two or more selected from among silane coupling-treated zinc oxide particles, silicone-treated zinc oxide particles, and sugar fatty acid ester-treated zinc oxide particles.

### Aspect 4

The composition according to Aspect 2 or 3,
wherein the surface-treated zinc oxide particles are one, two or more selected from among triethoxycaprylylsilane-treated zinc oxide, dimethicone- and hydrogen dimethicone-treated zinc oxide, and dextrin palmitate-treated zinc oxide particles.

### Aspect 5

The composition according to any one of Aspects 2 to 4,
wherein the surface of the surface-treated titanium oxide particles is treated with a fatty acid.

### Aspect 6

The composition according to any one of Aspects 1 to 5,
wherein a content of the UV scattering agent particles is 10 mass % or more.

### Aspect 7

The composition according to any one of Aspects 1 to 6, further including an aqueous component.

### Aspect 8

The composition according to any one of Aspects 1 to 7, further including a dispersing agent,
wherein a content of the dispersing agent is 0.5 to 10 mass %.

### Aspect 9

The composition according to Aspect 8,
wherein the dispersing agent contains polyhydroxystearic acid.

### Aspect 10

The composition according to any one of Aspects 1 to 9,
wherein a content of bis-diglyceryl polyacyladipate-2 is 2.0 to 5.0 mass %.

### Aspect 11

The composition according to any one of Aspects 1 to 10,
wherein the liquid oil is one, two or more selected from among alkyl fatty acid esters, triglycerides, vegetable oils, and hydrocarbon oils.

### Aspect 12

The composition according to any one of Aspects 1 to 11, which is a water-in-oil type emulsion composition.

### Aspect 13

The composition according to any one of Aspects 1 to 11, which is a sunscreen cosmetic composition.

### Advantageous Effects of Invention

According to the present invention, it is possible to provide a cosmetic composition having high water resistance even when no silicone oil or only a small amount thereof is used.

### DESCRIPTION OF EMBODIMENTS

Hereinafter, embodiments of the present invention will be described in detail. It should be noted that the present invention is not limited to the following embodiments, and various modifications can be performed within the scope of the gist of the invention.

### Cosmetic Composition

The cosmetic composition of the present invention (hereinafter simply referred to as a “composition of the present invention”) contains the following components:
UV scattering agent particles,
bis-diglyceryl polyacyladipate-2, and
a liquid oil,
wherein a content of a silicone oil in the liquid oil is 10 mass % or less.

As a result of extensive studies carried out by the inventors, problem was found in that, in a cosmetic composition such as sunscreen, when a silicone oil is not used as a liquid oil or only a small amount of a silicone oil is used, the cosmetic composition has poor water resistance.

In order to address this problem, the inventors conducted further studies in order to improve the water resistance of the cosmetic composition. As a result, when bis-diglyceryl polyacyladipate-2 is added to the cosmetic composition, the water resistance of the cosmetic composition is greatly improved.

In this case, bis-diglyceryl polyacyladipate-2 is sometimes added to the cosmetic composition as in, for example, the above PTL 2 and PTL 3. However, bis-diglyceryl polyacyladipate-2 in PTL 2 and PTL 3 is used in combination with a silicone oil, and is not a component for improving the water resistance of the cosmetic composition as in the present invention.

### UV Scattering Agent Particles

The composition of the present invention contains UV scattering agent particles. The UV scattering agent particles are metal particles having a UV protection effect (particularly metal oxide particles).

In the present invention, the average primary particle size of the UV scattering agent particles is not particularly limited, and may be, for example, 5 nm or more, 10 nm or more, or 15 nm or more, and may be 200 nm or less, 100 nm or less, or 50 nm or less. In this case, in the present invention, unless otherwise specified, the “average primary particle size” of particles may be determined as a diameter of a circle equivalent to the projected area of primary particles in a TEM image.

In addition, in the present invention, the shape of the UV scattering agent particle is not particularly limited, and examples thereof include a spherical shape, a plate shape, a rod shape, a spindle shape, a needle shape, and an irregular shape.

In the composition of the present invention, the content of the UV scattering agent particles is not particularly limited, and may be, for example, 10 mass % or more, 15 mass % or more, 20 mass % or more, 25 mass % or more, 30 mass % or more, or 34 mass % or more, and may be 70 mass % or less, 65 mass % or less, 60 mass % or less, 55 mass % or less, 50 mass % or less, 45 mass % or less, 40 mass % or less, or 35 mass % or less with respect to the entire composition.

In the composition of the present invention, the UV scattering agent particles can be dispersed in the liquid oil. In order to obtain a higher SPF, the amount of the UV scattering agent particles added may be 30 parts by mass or more, 35 parts by mass or more, 40 parts by mass or more, 45 parts by mass or more, 50 parts by mass or more, 55 parts by mass or more, or 60 parts by mass or more based on a total amount of 100 parts by mass of the liquid oil. In addition, the upper limit of the amount of the UV scattering agent particles added is not particularly limited, and may be, for example, 200 parts by mass or less, 150 parts by mass or less, or 100 parts by mass or less based on a total amount of 100 parts by mass of the liquid oil.

In the present invention, the UV scattering agent particles are not particularly limited as long as they are particles having a UV protection effect, and may be, for example, a combination of surface-treated zinc oxide particles and surface-treated titanium oxide particles.

### Surface-Treated Zinc Oxide Particles

In the present invention, the surface-treated zinc oxide particles are not particularly limited, and examples thereof include sugar fatty acid ester-treated zinc oxide particles, silane coupling agent-treated zinc oxide particles, silicone-treated zinc oxide particles, and stearic acid-treated zinc oxide particles.

The sugar fatty acid ester-treated zinc oxide particles are not particularly limited, and examples thereof include dextrin palmitate-treated zinc oxide particles, dextrin myristate-treated zinc oxide particles, dextrin (palmitate/hexyldecanoate)-treated zinc oxide particles, and dextrin (palmitate/ethylhexanoate)-treated zinc oxide particles, and among these, dextrin palmitate-treated zinc oxide particles are preferable.

The silane coupling agent-treated zinc oxide particles are not particularly limited, and examples thereof include zinc oxide particles treated with the following silane coupling agent. The silane coupling agent is not particularly limited, and examples thereof include trichlorosilanes such as octadecyltrichlorosilane, triethoxysilanes such as octyltriethoxysilane (OTS), and perfluoroalkylsilanes, and among these, octyltriethoxysilane-treated zinc oxide particles (that is, triethoxycaprylylsilane-treated zinc oxide particles) are preferable.

The silicone-treated zinc oxide particles are not particularly limited, and examples thereof include zinc oxide particles treated with the following silicone treatment agent. The silicone treatment agent is not particularly limited, and examples thereof include dimethylpolysiloxane (i.e., dimethicone), methylhydrogenpolysiloxane, a combination of methylhydrogenpolysiloxane and dimethylpolysiloxane, for example, a combination of dimethicone and hydrogen dimethicone and the like, but the present invention is not limited thereto.

As the surface-treated zinc oxide particles, commercially available particles may be used without change, or those obtained by treating the surface of zinc oxide particles with a surface treatment agent may be used.

For example, when the surface-treated zinc oxide particles are dextrin palmitate-treated zinc oxide particles, the zinc oxide particles can be mixed and stirred into a dextrin palmitate solution for a certain time and then filtered to obtain dextrin palmitate-treated zinc oxide particles.

In the present invention, the average primary particle size of the surface-treated zinc oxide particles is not particularly limited, and may be, for example, 5 nm or more, 10 nm or more, or 15 nm or more, and may be 200 nm or less, 100 nm or less, or 50 nm or less.

The shape of the surface-treated zinc oxide particle is not particularly limited, and examples thereof include a spherical shape, a plate shape, a rod shape, a spindle shape, a needle shape, and an irregular shape.

When the composition of the present invention contains surface-treated zinc oxide particles, the content thereof (a total content when the composition contains a mixture of a plurality of surface-treated zinc oxide particles) is not particularly limited, and may be, for example, 5.0 mass % or more, 10 mass % or more, 15 mass % or more, 18 mass % or more, or 20 mass % or more, and may be 50 mass % or less, 45 mass % or less, 40 mass % or less, 35 mass % or less, 30 mass % or less, 25 mass % or less, or 20 mass % or less with respect to the entire composition.

### Surface-Treated Titanium Oxide Particles

In the present invention, the surface treatment may be, for example, a surface treatment for hydrophobization.

More specifically, examples of surface treatments for titanium oxide particles include a fatty acid treatment (including a metal soap treatment), a fluorine compound treatment, a pendant treatment, a silane coupling agent treatment, a silicone treatment, a titanium coupling agent treatment, an oiling agent treatment, an N-acylated lysine treatment, a polyacrylic acid treatment, an amino acid treatment, an inorganic compound treatment, a plasma treatment, and a mechanochemical treatment, but the present invention is not limited thereto. Among these surface treatments, in consideration of dispersion stability and the like, a fatty acid treatment, particularly, a stearic acid treatment, a stearic acid soap treatment and the like are preferable. Examples of stearic acid soap treatments include a treatment with a metal salt of stearic acid or a mixture of stearic acid and a metal hydroxide (for example, aluminum hydroxide, etc.), but the present invention is not limited thereto. In addition, in order to inhibit the catalytic activity of titanium oxide, it is preferable to subject the titanium oxide particles to a surface treatment with an inorganic compound, for example, aluminum hydroxide, before a surface treatment with an organic compound such as a fatty acid treatment. Therefore, in a preferable aspect, the surface of the titanium oxide particles is treated with, for example, aluminum hydroxide and fatty acids.

As the surface-treated titanium oxide particles, commercially available particles may be used without change, and those obtained by treating the surface of titanium oxide particles with the above surface treatment agent.

In the present invention, the average primary particle size of the surface-treated titanium oxide particles is not particularly limited, and may be, for example, 5 nm or more, 10 nm or more or 15 nm or more, and may be 200 nm or less, 100 nm or less, or 50 nm or less.

The shape of the surface-treated titanium oxide particle is not particularly limited, and examples thereof include a spherical shape, a plate shape, a rod shape, a spindle shape, a needle shape, and an irregular shape.

When the composition of the present invention contains surface-treated titanium oxide particles, the content thereof is not particularly limited, and may be, for example, 5.0 mass % or more, 6.0 mass % or more, 7.0 mass % or more, 8.0 mass % or more, 9.0 mass % or more, 10 mass % or more, or 13 mass % or more, and may be 20 mass % or less, 15 mass % or less, or 13 mass % or less with respect to the entire composition.

In addition, when the UV scattering agent particles are a combination of surface-treated zinc oxide particles and surface-treated titanium oxide particles, the relationship between the content of surface-treated zinc oxide particles in the composition of the present invention and the content of the surface-treated titanium oxide particles in the composition of the present invention is not particularly limited, and the content of the surface-treated zinc oxide particles (a total content when the composition contains a mixture of fatty acid-treated zinc oxide particles and fatty acid ester-treated zinc oxide particles) is preferably larger than the content of the surface-treated titanium oxide particles.

### Bis-Diglyceryl Polyacyladipate-2

The composition of the present invention contains bis-diglyceryl polyacyladipate-2. When bis-diglyceryl polyacyladipate-2 is contained, it is possible to impart high water resistance to the composition of the present invention even when no silicone oil or only a small amount thereof is used.

Bis-diglyceryl polyacyladipate-2 is an ester oil of diglycerol, and adipic acid, octanoic acid, decanoic acid, isostearic acid, stearic acid, or hydroxystearic acid. Since, in the present invention, bis-diglyceryl polyacyladipate-2 is a semi-solid oil, that is, an oil that is not completely solidified or liquefied at room temperature (25° C.) under atmospheric pressure (1 atm (9.8×10⁴ Pa)), it is distinguished from a liquid oil and a solid oil, and is classified as an oil having a melting point in a range of, for example, higher than 25° C. (preferably 30° C. or higher) and 55° C. or lower (preferably 50° C. or lower). In this case, in the present invention, “solid” refers to a substance that can maintain its solid state at room temperature (25° C.) under atmospheric pressure (1 atm (9.8×10⁴ Pa)).

In the composition of the present invention, the content of bis-diglyceryl polyacyladipate-2 is not particularly limited, and may be, for example, 2.0 mass % or more, 2.5 mass % or more, or 3.0 mass % or more, and 5.0 mass % or less, 4.5 mass % or less, 4.0 mass % or less, 3.5 mass % or less, or 3.0 mass % or less with respect to the entire composition.

### Liquid Oil

The composition of the present invention contains a liquid oil. The liquid oil refers an oil having fluidity at room temperature (25° C.) under atmospheric pressure (1 atm (9.8×10⁴ Pa)), and may be, for example, an oil having a melting point of 25° C. or lower (preferably lower than 25° C., 20° C. or lower, 15° C. or lower, or 10° C. or lower).

In the present invention, the liquid oil is not particularly limited, but in order to make the composition silicone-free or reduce the amount of silicone used and in order to better exhibit the effect of the present invention, it is preferable that the liquid oil do not contain a silicone oil. Alternatively, when the liquid oil according to the present invention contains a silicone oil, the content thereof is preferably 10 mass % or less, 9.0 mass % or less, 8.0 mass % or less, 7.0 mass % or less, 6.0 mass % or less, 5.0 mass % or less, 4.0 mass % or less, 3.0 mass % or less, 2.0 mass % or less, 1.0 mass % or less, 0.5 mass % or less, 0.1 mass % or less, or 0.01 mass % or less based on a total mass of the liquid oil.

The liquid oil according to the present invention may be, for example, one, two or more selected from among alkyl fatty acid esters, triglycerides, vegetable oils, and hydrocarbon oils.

More specifically, examples of alkyl fatty acid esters include palmitates such as octyl palmitate, octanoates such as cetyl octanoate, isooctanoates such as glyceryl tri-2-ethylhexanoate, and pentaerythritol tetra-2-ethylhexanoate, laurates such as hexyl laurate, myristates such as isopropyl myristate and octyldodecyl myristate, stearates such as isocetyl stearate, isostearates such as isopropyl isostearate, isopalmitates such as isooctyl palmitate, oleates such as isodecyl oleate, adipic acid diesters such as diisopropyl adipate, sebacic acid diesters such as diethyl sebacate, and ester oils such as diisostearyl malate, but the present invention is not limited thereto.

Examples of triglycerides include tricaprylin, tricaproin, triisostearin, trielaidin, trierucin, trilinolein, triolein, caprylic/capric triglyceride, caprylic/capric/linoleic triglyceride, and ricinoleic/caproic/caprylic/capric triglyceride, but the present invention is not limited thereto.

Examples of vegetable oils include jojoba seed oil, olive oil, macadamia nut oil, meadowfoam oil, castor oil, safflower oil, sunflower oil, avocado oil, canola oil, apricot kernel oil, rice germ oil, and rice bran oil, but the present invention is not limited thereto.

Examples of hydrocarbon oils include branched hydrocarbon oils such as squalane, isododecane, and isohexadecane, and linear hydrocarbon oils such as undecane and tridecane, but the present invention is not limited thereto.

In the composition of the present invention, the content of the liquid oil is not particularly limited, and may be, for example, 10 mass % or more, 20 mass % or more, 30 mass % or more, 40 mass % or more, 50 mass % or more, or 60 mass % or more, and may be 90 mass % or less, 80 mass % or less, or 60 mass % or less with respect to the entire composition.

### Optional Components

The composition of the present invention may further contain one or more optional components. The optional components are not particularly limited, and examples thereof include a dispersing agent, an organically modified clay mineral, and an aqueous component.

### Dispersing Agent

The composition of the present invention may further contain, optionally, a dispersing agent. The dispersing agent mainly has a function of uniformly dispersing UV scattering agent particles in a liquid oil and has an effect of imparting spreadability to the cosmetic composition.

When the composition of the present invention contains a dispersing agent, the content thereof is not particularly limited, and may be, for example, 0.5 mass % or more, 1.0 mass % or more, 1.5 mass % or more, 2.0 mass % or more, 2.5 mass % or more, 3.0 mass % or more, or 3.3 mass % or more, and may be 10 mass % or less, 9.0 mass % or less, 8.0 mass % or less, 7.0 mass % or less, 6.0 mass % or less, 5.0 mass % or less, or 4.0 mass % or less with respect to the entire composition.

The dispersing agent is not particularly limited, and examples thereof include polyhydroxystearic acid, polyricinoleic acid polyglycerol ester, isostearic acid, polyglyceryl-2 diisostearate, and PEG-10 dimethicone. Among these, in consideration of silicone-free, the dispersing agent preferably contains one or more selected from among polyhydroxystearic acid, polyricinoleic acid polyglycerol ester, isostearic acid, and polyglyceryl-2 diisostearate.

In addition, in order to improve transparency upon application, the dispersing agent preferably contains polyhydroxystearic acid and polyricinoleic acid polyglycerol ester, and particularly preferably contains polyhydroxystearic acid. In this case, polyhydroxystearic acid is a compound oligomerized by forming ester bonds with hydroxystearic acid, commercially available products include, for example, HS oligomer 600 (commercially available from Hokoku Corporation), and Salacos HS-6C (commercially available from The Nisshin OilliO Group, Ltd.), and these can be used. In addition, the degree of polymerization of polyhydroxystearic acid is not particularly limited, and may be, for example, 4 to 8.

### Organically Modified Clay Mineral

The composition of the present invention may further contain, optionally, an organically modified clay mineral. For example, when the cosmetic composition is a water-in-oil type emulsion composition, the organically modified clay mineral has an effect of improving the caking resistance of the cosmetic composition.

The organically modified clay mineral is a type of colloidal hydrous aluminum silicate having a three-layer structure, and a typical example thereof is a clay mineral represented by the following General Formula (1) modified with a quaternary ammonium salt type cationic surfactant. (X, Y)₂₋₃(Si, Al)₄O₁₀(OH)₂Z_{1/3}·nH₂O (1)

In Formula (1), X is Al, Fe (III), Mn (III), or Cr (III), Y is Mg, Fe (II), Ni, Zn, or Li, and Z is K, Na, or Ca.

Specific examples of organically modified clay minerals include dimethyl distearammonium hectorite (disteardimonium hectorite), dimethyl alkyl ammonium hectorite, benzyl dimethyl stearyl ammonium hectorite, and distearyl dimethyl ammonium chloride-treated magnesium aluminum silicate, but the present invention is not limited thereto. Among these, the organically modified clay mineral is preferably disteardimonium hectorite.

In addition, as commercially available organically modified clay minerals, for example, Bentone 27 (benzyl dimethyl stearyl ammonium chloride-treated hectorite: commercially available from Elementis Japan Co., Ltd.), Bentone 38 (distearyl dimethyl ammonium chloride-treated hectorite: commercially available from Elementis Japan Co., Ltd.) and the like can be used.

When the composition of the present invention contains an organically modified clay mineral, the content thereof is not particularly limited, and may be, for example, 0.05 mass % or more, 0.1 mass % or more, 0.2 mass % or more, 0.3 mass % or more, 0.4 mass % or more, or 0.5 mass % or more, and may be 10 mass % or less, 5.0 mass % or less, or 1.0 mass % or less with respect to the entire composition.

### Aqueous Component

The composition of the present invention may further contain, optionally, an aqueous component. The aqueous component can impart a refreshing effect to the composition of the present invention.

The aqueous component is not particularly limited, and for example, water, monohydric alcohols such as ethanol, polyhydric alcohols such as butylene glycol, dipropylene glycol, propanediol, pentylene glycol, hexanediol, and glycerin, polyalkylene glycol such as polyethylene glycol and polypropylene glycol or copolymers thereof, inorganic salts such as sodium chloride and magnesium chloride, or mixtures thereof can be used.

When the composition of the present invention contains an aqueous component, the content thereof is not particularly limited, and may be, for example, 0.5 mass % or more, 1.0 mass % or more, 2.0 mass % or more, 3.0 mass % or more, 4.0 mass % or more, 5.0 mass % or more, 6.0 mass % or more, 7.0 mass % or more, 8.0 mass % or more, 9.0 mass % or more, or 10 mass % or more, and may be 90 mass % or less, 80 mass % or less, 70 mass % or less, 60 mass % or less, 50 mass % or less, 40 mass % or less, 30 mass % or less, 20 mass % or less, 10 mass % or less, or 5.0 mass % or less with respect to the entire composition. In this case, the content of the aqueous component may be appropriately adjusted based on the formulation of the composition of the present invention.

### Other Components

In addition to the above components, the composition of the present invention may further contain, optionally, for example, alcohols, moisturizing agents, thickeners, powder components (powders other than the above UV scattering agent particles), antioxidants, stabilizing agents, chelating agents, preservatives, and fragrance agents.

### Formulation and Use

The formulation of the cosmetic composition of the present invention is not particularly limited. Therefore, the cosmetic composition of the present invention may be a water-in-oil type emulsion cosmetic composition, an oil-in-water type emulsion cosmetic composition, an oily cosmetic composition based on an oiling agent, or the like.

The composition of the present invention can be used as a sunscreen cosmetic. More specifically, the composition of the present invention can be provided as, for example, a sunscreen cream, a sunscreen emulsion, or a sunscreen lotion, and can also be used as a foundation, a makeup base, a makeup cosmetic, a hair cosmetic or the like with a sunscreen effect imparted.

The method of preparing the composition of the present invention is not particularly limited, and the composition can be prepared according to a method generally used depending on a desired formulation. For example, when the composition of the present invention is a water-in-oil type emulsion cosmetic composition, it can be prepared by separately preparing an oil phase component obtained by dispersing the above UV scattering agent particles in a liquid oil and an aqueous component, adding the aqueous component while stirring the oil phase component, and performing emulsifying.

The present invention will be described below in more detail with reference to examples, but the present invention is not limited thereto. In this case, unless otherwise specified, the addition amount is mass % based on the mass of the cosmetic composition.

### Examples 1 and 2 and Comparative Examples 1 to 5

Based on the formulations shown in the following Table 1 and Table 2, cosmetic compositions of examples and comparative examples were prepared. The obtained compositions were evaluated for “water resistance” according to the following criteria, and the results are shown in Table 1.

### Water Resistance Evaluation

A certain amount (2 mg/cm²) of the composition was added dropwise to a 50 mm square acrylic resin pseudo-substrate simulating the texture of human skin, and the composition was uniformly applied onto the substrate with a certain load (100±30 g) at a certain speed (100 mm/sec×50 reciprocations). After drying for a certain time (15 minutes), a spectrophotometer (SPECTROPHOTOMETER U-3500, commercially available from Hitachi, Ltd.) was used to transmit UV light and visible light of 280 to 500 nm through the coated substrate, and the absorbance was calculated from the transmittance measurement. In addition, the coated substrate was immersed in a water flow generated by propeller stirring for 30 minutes, and the transmittance was measured again by the above method after drying. The ratio of the integrated absorbance at 280 to 340 nm before and after the test of immersion in water was set as the water resistance rate, and evaluated according to the following criteria:
A: the water resistance rate was 70% or more
B: the water resistance rate was 50% or more and less than 70%
C: the water resistance rate was 30% or more and less than 50%
D: the water resistance rate was less than 30%

TABLE 1
(Examples 1 and 2, and Comparative Examples 1 to 5)

| | | | | Com- | Com- | Com- | Com- | Com- |
|---|---|---|---|---|---|---|---|---|
| | | Exam- | Exam- | parative | parative | parative | parative | parative |
| Constituent components | | ple 1 | ple 2 | Example 1 | Example 2 | Example 3 | Example 4 | Example 5 |
| UV scattering | Surface-treated titanium | 13 | 13 | 13 | 13 | 13 | 13 | 13 |
| agent particles | oxide particles | | | | | | | |
| | (stearic acid/aluminum | | | | | | | |
| | hydroxide/titanium oxide) | | | | | | | |
| | Surface-treated zinc oxide | 20 | 20 | 20 | 20 | 20 | 20 | 20 |
| | particles | | | | | | | |
| | (dextrin palmitate/zinc | | | | | | | |
| | oxide) | | | | | | | |
| Dispersing agent | Polyhydroxystearic acid | 3.3 | 3.3 | 3.3 | 3.3 | 3.3 | 3.3 | 3.3 |
| Bis-diglyceryl polyacyladipate-2 | | 3.0 | 2.0 | — | — | — | — | — |
| Diisostearyl malate | | — | — | — | 3.0 | — | — | — |
| Di (phytosteryl/octyldodecyl) lauroyl glutamate | | — | — | — | — | 3.0 | — | — |
| Macadamia nut fatty acid phytosteryl | | — | — | — | — | — | 3.0 | — |
| Pentaerythrityl tetra (behenate/benzoate/ | | — | — | — | — | — | — | 3.0 |
| ethylhexanoate) | | | | | | | | |
| Aqueous component | Water | 1.0 | 1.0 | 1.0 | 1.0 | 1.0 | 1.0 | 1.0 |
| | Glycerin | 1.0 | 1.0 | 1.0 | 1.0 | 1.0 | 1.0 | 1.0 |
| Organically | Distearyldimonium hectorite | 0.5 | 0.5 | 0.5 | 0.5 | 0.5 | 0.5 | 0.5 |
| modified clay | | | | | | | | |
| mineral | | | | | | | | |
| Liquid oil | Details are shown in Table 2 | remainder | remainder | remainder | remainder | remainder | remainder | remainder |
| Sum | | 100 | 100 | 100 | 100 | 100 | 100 | 100 |
| Evaluation | Water resistance | A | A | D | D | D | D | D |

TABLE 2
(Table 2 Details of “liquid oil” in Table 1 and Table 3)

| | Addition amount |
|---|---|
| | (mass % based on total |
| Liquid oil | mass of liquid oil) |

| | | |
|---|---|---|
| Vegetable oil | Jojoba seed oil | remainder |
| Branched hydrocarbon | Squalane | 20 |
| oil | | |
| Triglyceride | Tricaprylin | 14 |
| Alkyl fatty acid | Octyl palmitate | 9.0 |
| ester | | |
| Linear hydrocarbon | Undecane | 12 |
| oil | Tridecane | 5.0 |
| Antioxidant | Tocopherol | appropriate amount |

| | |
|---|---|
| Sum | 100 |

As is clear from the results in Table 1, it can be understood that the cosmetic compositions of Examples 1 and 2 both had excellent water resistance in the evaluation results.

On the other hand, it can be understood that the cosmetic composition of Comparative Example 1 containing no bis-diglyceryl polyacyladipate-2, and the cosmetic compositions of Comparative Examples 2 to 5 in which diisostearyl malate, di(phytosteryl/octyldodecyl) lauroyl glutamate, macadamia nut fatty acid phytosteryl, and pentaerythrityl tetra(behenate/benzoate/ethylhexanoate) were added in place of bis-diglyceryl polyacyladipate-2 all had poor water resistance in the evaluation results.

In this case, diisostearyl malate, di(phytosteryl/octyldodecyl) lauroyl glutamate, macadamia nut fatty acid phytosteryl, pentaerythrityl tetra(behenate/benzoate/ethylhexanoate) and the like are oils used as cosmetic additive components, and all of them have a function of a hydrated oiling agent like bis-diglyceryl polyacyladipate-2.

### Examples 3 to 9

Based on the formulations shown in the following Table 3, cosmetic compositions of Examples 3 to 9 were prepared. The obtained compositions were evaluated for “water resistance” in the same manner as above, and the results are shown in Table 3.

In addition, the transparency when each cosmetic composition was applied was evaluated as follows, and the results are shown in Table 3.

### Transparency Evaluation

The compositions obtained in the examples were subjected to a usage test by 20 cosmetic evaluation specialist panelists. Each panelist evaluated “transparency” for the compositions on a 5-point scale according to the following “evaluation criteria,” and additionally, the average score of the scores of all the panelists was determined according to the following 4-point scale determination criteria.

### Evaluation Criteria

evaluation results: score
very good: 5 points
good: 4 points
average: 3 points
slightly poor: 2 points
poor: 1 point

### Determination Criteria

“A”: the average score was 4 or more
“B”: the average score was 3 or more and less than 4
“C”: the average score was 1.5 or more and less than 3
“D”: the average score was less than 1.5

TABLE 3
(Examples 3 to 9)

| | | Exam- | Exam- | Exam- | Exam- | Exam- | Exam- | Exam- |
|---|---|---|---|---|---|---|---|---|
| Constituent components | | ple 3 | ple 4 | ple 5 | ple 6 | ple 7 | ple 8 | ple 9 |
| UV scattering | Surface-treated titanium | 13 | 13 | 13 | 13 | 13 | 13 | 13 |
| agent particles | oxide particles | | | | | | | |
| | (stearic acid/aluminum | | | | | | | |
| | hydroxide/titanium oxide) | | | | | | | |
| | Surface-treated zinc | 20 | — | — | 20 | 20 | 20 | 20 |
| | oxide particles | | | | | | | |
| | (dextrin palmitate/zinc oxide) | | | | | | | |
| | Surface-treated zinc oxide | — | 20 | — | — | — | — | — |
| | (triethoxycaprylylsilane/ | | | | | | | |
| | zinc oxide) | | | | | | | |
| | Surface-treated zinc oxide | — | — | 20 | — | — | — | — |
| | (hydrogen dimethicone/ | | | | | | | |
| | zinc oxide) | | | | | | | |
| Dispersing agent | Polyhydroxystearic acid | 3.3 | 3.3 | 3.3 | — | — | — | — |
| | Polyglyceryl-6 polyricinoleate | — | — | — | 3.3 | — | — | — |
| | Isostearic acid | — | — | — | — | 3.3 | — | — |
| | Polyglyceryl-2 diisostearate | — | — | — | — | — | 3.3 | — |
| | PEG-10 dimethicone | — | — | — | — | — | — | 3.3 |
| Bis-diglyceryl polyacyladipate-2 | | 3.0 | 3.0 | 3.0 | 3.0 | 3.0 | 3.0 | 3.0 |
| Aqueous | Water | 1.0 | 1.0 | 1.0 | 1.0 | 1.0 | 1.0 | 1.0 |
| component | Glycerin | 1.0 | 1.0 | 1.0 | 1.0 | 1.0 | 1.0 | 1.0 |
| Organically | Distearyldimonium hectorite | 0.5 | 0.5 | 0.5 | 0.5 | 0.5 | 0.5 | 0.5 |
| modified | | | | | | | | |
| clay mineral | | | | | | | | |
| Liquid oil | Details are shown in Table 2 | remainder | remainder | remainder | remainder | remainder | remainder | remainder |
| Sum | | 100 | 100 | 100 | 100 | 100 | 100 | 100 |
| Evaluation | Water resistance | A | A | A | A | A | A | A |
| | Transparency | A | A | A | A | C | C | C |
| | upon application | | | | | | | |

As is clear from the results in Table 3, it can be understood that the cosmetic compositions of Examples 3 to 9 all had excellent water resistance in the evaluation results.

In addition, it can be understood that, in the evaluation of transparency upon application, the cosmetic compositions of Examples 7 to 9 were slightly poor, but the cosmetic compositions of Examples 3 to 6 were all excellent. In this case, although not shown in Table 3, the cosmetic compositions of Examples 1 and 2 were also similarly evaluated for the transparency upon application, and all were evaluated as “A.”

1. A cosmetic composition comprising the following components:
UV scattering agent particles,
bis-diglyceryl polyacyladipate-2, and
a liquid oil,
wherein a content of a silicone oil in the liquid oil is 10 mass % or less.

2. The composition according to claim 1,
wherein the UV scattering agent particles are a combination of surface-treated zinc oxide particles and surface-treated titanium oxide particles.

3. The composition according to claim 2,
wherein the surface-treated zinc oxide particles are one, two or more selected from among sugar fatty acid ester-treated zinc oxide particles, silane coupling agent-treated zinc oxide particles, and silicone-treated zinc oxide particles.

4. The composition according to claim 3,
wherein the surface-treated zinc oxide particles are one, two or more selected from among dextrin palmitate-treated zinc oxide particles, triethoxycaprylylsilane-treated zinc oxide particles, and dimethicone- and hydrogen dimethicone-treated zinc oxide particles.

5. The composition according to claim 2,
wherein the surface of the surface-treated titanium oxide particles is treated with a fatty acid.

6. The composition according to claim 1,
wherein a content of the UV scattering agent particles is 10 mass % or more.

7. The composition according to claim 1, further comprising an aqueous component.

8. The composition according to claim 1, further comprising a dispersing agent,
wherein a content of the dispersing agent is 0.5 to 10 mass %.

9. The composition according to claim 8,
wherein the dispersing agent contains polyhydroxystearic acid.

10. The composition according to claim 1,
wherein a content of bis-diglyceryl polyacyladipate-2 is 2.0 to 5.0 mass %.

11. The composition according to claim 1,
wherein the liquid oil is one, two or more selected from among alkyl fatty acid esters, triglycerides, vegetable oils, and hydrocarbon oils.

12. The composition according to claim 1, which is a water-in-oil type emulsion composition.

13. The composition according to claim 1, which is a sunscreen cosmetic composition.
